(19) United States
(12) Patent Application Publication
(10) Pub. No.: US 2016/0003781 A1
(43) Pub. Date: Jan. 7, 2016

(54) OBJECT INFORMATION ACQUIRING APPARATUS AND LASER APPARATUS

(71) Applicant: CANON KABUSHIKI KAISHA, Tokyo (JP)

(72) Inventors: Shigeru Ichihara, Tokyo (JP); Minoru Ohkoba, Sagamihara-shi (JP)

(21) Appl. No.: 14/751,330

(22) Filed: Jun. 26, 2015

(30) Foreign Application Priority Data
Jul. 3, 2014 (JP) 2014-137597

(51) Int. Cl. G01N 29/24; H01S 3/091; H01S 3/115; G01N 29/04; G01N 33/483

(52) U.S. Cl. CPC G01N 29/2418; G01N 29/04; G01N 33/4833; H01S 3/115; H01S 3/091; G01N 2291/023; G01N 2291/02809

(57) ABSTRACT

There is used an object information acquiring apparatus including a laser resonator configured to include two reflection bodies and a Q switch provided between the two reflection bodies and determining a Q value based on a voltage to be applied, an excitation section configured to optically excite a laser medium, a detector configured to detect light output from one of the reflection bodies, a controller configured to control the laser resonator and determine a value of the voltage based on a detection result of the detector and applies the voltage to the Q switch, a receiver configured to receive an acoustic wave propagating from an object based on irradiation of the object with laser light output from one of the reflection bodies, and an acquisition section configured to acquire information on the object based on a reception result of the receiver.

### BACKGROUND OF THE INVENTION

1. Field of the Invention

The present invention relates to an object information acquiring apparatus and a laser apparatus.

2. Description of the Related Art

Photoacoustic tomography (PAT) is a method in which a segment to be measured is irradiated with a light flux by using a pulse laser as a light source, a photoacoustic wave generated by the irradiation is received by a probe and subjected to signal processing, and image formation (including image reconstruction) is performed. It is possible to analyze biological functions by using laser light having a wavelength corresponding to the absorption spectrum of a biological tissue.

The light flux having entered a biological object is rapidly diffused inside the biological body, and hence a high energy output and the laser light having a short pulse width in order to obtain a large photoacoustic wave from the biological tissue are required. As the laser for a photoacoustic diagnostic apparatus, a Q-switched pulse laser that uses a Pockels cell is suitable. An alexandrite laser can variably control its wavelength, has a long fluorescence life of a laser medium, and is capable of direct excitation by a flash lamp. Accordingly, the alexandrite laser is suitably used for the above biological function analysis.

The flash lamp that excites the laser medium has an advantage that high-output laser oscillation can be obtained easily and inexpensively, but light energy of excitation light is converted to thermal energy and the temperature of the internal portion of a resonator is thereby increased. The change of the temperature of the internal portion of the resonator influences the alignment of the resonator and a polarization characteristic of the Pockels cell. As a result, there is a possibility that prelasing oscillation occurs. To cope with this point, a method in which prelasing light is detected and oscillation control is performed is proposed (U.S. Pat. No. 5,355,383).

### SUMMARY OF THE INVENTION

U.S. Pat. No. 5,355,383 discloses a method in which the prelasing light that influences stable oscillation is detected and laser oscillation is stopped based on the detection. However, in an object information acquiring apparatus, it is desirable to stably use a laser apparatus while suppressing the occurrence of prelasing.

In view of the foregoing, an object of the present invention is to provide a technique for performing stable giant pulse oscillation by reducing the occurrence of the prelasing.

In order to achieve the above object, the present invention adopts the following configuration. That is, the present invention adopts an object information acquiring apparatus comprising: a laser resonator configured to include two reflection bodies and a Q switch provided between the two reflection bodies and determining a Q value based on a voltage to be applied; an excitation section configured to optically excite a laser medium; a detector configured to detect light output from one of the reflection bodies; a controller configured to control the laser resonator and determine a value of the voltage based on a detection result of the detector and applies the voltage to the Q switch; a receiver configured to receive an acoustic wave propagating from an object based on irradiation of the object with laser light output from one of the reflection bodies; and
an acquisition section configured to acquire information on the object based on a reception result of the receiver.

The present invention also adopts the following configuration. That is, the present invention adopts a laser apparatus comprising: a laser resonator configured to include two reflection bodies and a Q switch provided between the two reflection bodies and determining a Q value based on a voltage to be applied; an excitation section configured to optically excite a laser medium; a detector configured to detect light output from one of the reflection bodies; and a controller configured to control the laser resonator and determine a value of the voltage based on a detection result of the detector and applies the voltage to the Q switch.

According to the present invention, in the laser apparatus, it is possible to provide the technique for performing the stable giant pulse oscillation by reducing the occurrence of the prelasing.

Further features of the present invention will become apparent from the following description of exemplary embodiments with reference to the attached drawings.

### BRIEF DESCRIPTION OF THE DRAWINGS

FIG. 1 is a block diagram showing a laser apparatus according to a comparative technique to the present invention;

FIGS. 2A to 2C are views each showing the relationship between a laser output and time during normal oscillation of the comparative technique;

FIGS. 3A to 3C are views each showing the relationship between the laser output and time during abnormal oscillation of the comparative technique;

FIG. 4 is a block diagram showing a first embodiment of a laser apparatus of the present invention;

FIG. 5 is a block diagram including the laser apparatus of the present invention and an object;

FIG. 6 is a block diagram showing a second embodiment of the laser apparatus of the present invention; and

FIG. 7 is a view showing a third embodiment of the laser apparatus of the present invention.

### DESCRIPTION OF THE EMBODIMENTS

Herein below, embodiments of the present invention will be described in detail with reference to the drawings. Note that, in principle, like elements are designated by like reference numerals and the description thereof will be omitted. However, detailed calculating formulas and calculation procedures, the dimension, material, shape, and relative arrangement of each component described below should be appropriately changed according to the configuration and various conditions of an apparatus to which the present invention is applied, and are not intended to limit the scope of the present invention to the following description.

An object information acquiring apparatus of the present invention includes an apparatus utilizing photoacoustic effects in which an acoustic wave generated inside an object by irradiating the object with light (electromagnetic wave) such as a near infrared ray or the like is received and object information is acquired as image data. In the case of the apparatus that utilizes the photoacoustic effects, the acquired object information includes a generation source distribution of the acoustic wave generated by the light irradiation, an initial sound pressure distribution inside the object, a light energy absorption density distribution or an absorption coefficient distribution derived from the initial sound pressure distribution, or a concentration distribution of a substance constituting a tissue. Examples of the concentration distribution of the substance include an oxygen saturation distribution, a total hemoglobin concentration distribution, and an oxidized-reduced hemoglobin concentration distribution.

Characteristic information as the object information at a plurality of positions may be acquired as a two-dimensional or three-dimensional characteristic distribution. The characteristic distribution can be generated as image data indicative of characteristic information on the inside of the object. The acoustic wave in the present invention is an ultrasonic wave typically, and includes waves called a sound wave and a photoultrasonic wave. The acoustic wave generated by the photoacoustic effects is called a photoacoustic wave or the photoultrasonic wave. An acoustic detector (e.g., a probe) receives the acoustic wave generated or reflected in the object.

In addition, an electric signal converted from the photoacoustic wave by a conversion element or a signal obtained by performing signal processing (amplification, AD conversion) or information processing on the electric signal is called a photoacoustic signal.

### <Comparative Technique>

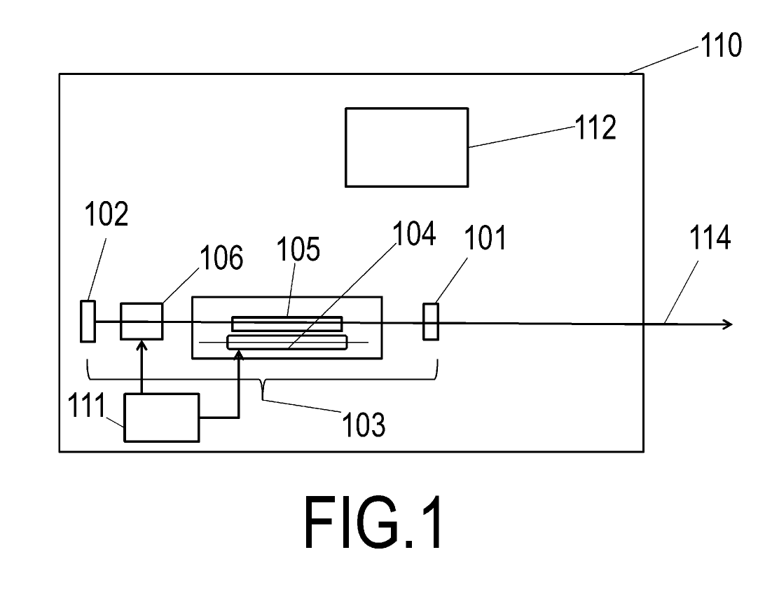

FIG. 1 is a block diagram showing a laser apparatus according to a comparative technique to the present invention. Features of a laser apparatus according to the present invention will become more apparent by comparison with a laser apparatus 110 according to the comparative technique. Therefore, the comparative technique will be described first. As shown in FIG. 1, the laser apparatus 110 according to the comparative technique includes a laser resonator 103 that includes an output mirror 101 and a reflection mirror 102 as two reflection bodies. Further, the laser apparatus 110 includes a laser controller 111 as a controller, and a laser power supply 112 that supplies power to the laser apparatus 110. Note that wiring of the laser power supply 112 or the like is omitted. The laser controller 111 is provided in the laser apparatus 110.

An excitation section (an excitation device) 104, a laser medium 105, and a Q switch 106 are disposed in the resonator 103. The laser controller 111 controls a voltage applied to the excitation section 104 and the Q switch 106. The excitation section 104 uses a flash lamp and a semiconductor laser and, in the case where the rod-shaped laser medium 105 is used, performs optical excitation from the side surface of the laser medium 105. In the Q switch 106, a Pockels cell as an optical crystal of potassium dihydrogen phosphate (KDP) or potassium dideuterium phosphate (DKDP) is used. The Pockels cell is an element in which its index of refraction changes in proportion to the intensity of an electric field, and a polarization direction of transmitted light rotates. Accordingly, it is widely used in order to obtain laser light 114 having a narrow oscillation pulse width and a strong output intensity. The pulse width differs depending on the type of the laser medium, the length of the resonator, and an optical resonance state, and the pulse width of not more than 100 ns is obtained. In the case where an Nd:YAG crystal or an alexandrite crystal is used in the laser medium, the configuration of FIG. 1 is used. On the other hand, in the case of a titanium sapphire laser, the second harmonic of an Nd:YAG laser is used as an excitation source of a titanium sapphire crystal. In the titanium sapphire laser, the present invention is applied to an Nd:YAG laser portion serving as the excitation source. Note that, although the laser apparatus in the following description will be described mainly by using an alexandrite laser that excites the laser medium using the flash lamp as a reference, the laser apparatus is not limited thereto, and other laser apparatuses may also be used.

The alexandrite laser has a gain in the range of 700 nm to 800 nm, and serves as a wavelength tunable laser by disposing a wavelength selection mechanism including a birefringent filter between the laser medium 105 and the Pockels cell, i.e., the Q switch 106 in the resonator 103.

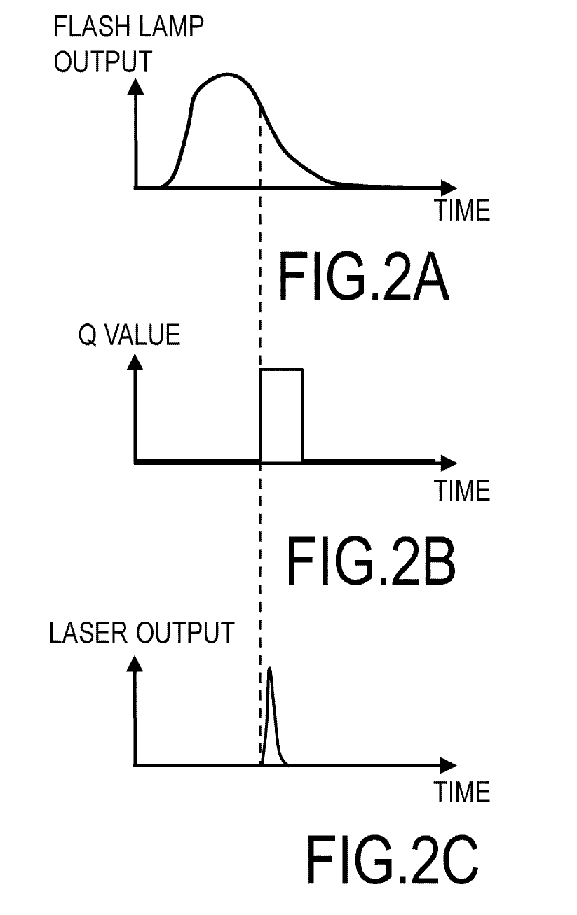

FIGS. 2A to 2C are views each showing the relationship between a laser output and time during normal oscillation of the comparative technique. By using FIGS. 2A to 2C, giant pulse oscillation that uses the Q switch will be described. FIG. 2A is a view in which the vertical axis indicates the output of the flash lamp, the horizontal axis indicates time, and the relationship between the output of the flash lamp and time is shown. As shown in FIG. 2A, in a state in which the Q switch 106 is ON and a Q value in the resonator 103 is maintained at a low value, the laser medium is excited and a population inversion energy density is increased. Next, as shown in FIG. 2B, the Q switch is turned OFF, and the Q value of the resonator is momentarily increased. Next, as shown in FIG. 2C, energy accumulated in the laser medium 105 optically resonates in the resonator 103, a laser oscillation threshold value is exceeded, and laser light 114 having a short pulse oscillates. The above laser light 114 is referred to as giant pulse oscillation. On the other hand, the laser light 114 having a temporal spread obtained by exciting the laser medium using excitation light in a state in which the Q value of the resonator is high is referred to as normal pulse oscillation. The giant pulse is the laser light having a pulse width of not more than 100 nanoseconds (ns). On the other hand, the normal pulse is dependent on a light emission time width of the excitation source, and has the pulse width of about several hundreds of microsecond (us) in the case where the flash lamp is used.

The laser apparatus 110 applies a voltage to the Pockels cell as the Q switch 106. By the application, the Q value of the resonator 103 is reduced (Low state in FIG. 2B). Thereafter, the application of the voltage is stopped. By stopping the application thereof, the Q value of the resonator 103 is increased (High state in FIG. 2B). In this state, the giant pulse is caused to oscillate. The technique has an advantage that it is possible to arbitrarily control the polarization state immediately before the giant pulse oscillation by changing the voltage applied to the Pockels cell.

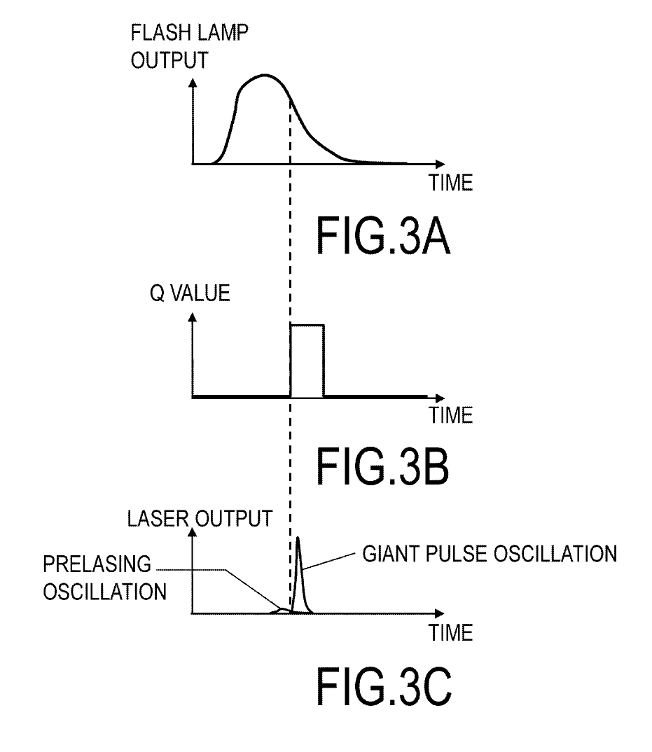

FIGS. 3A to 3C are views each showing the relationship between the laser output and time during abnormal oscillation of the comparative technique. Prelasing oscillation will be described by using FIGS. 3A to 3C. FIGS. 3A and 3B are the same as FIGS. 2A and 2B, respectively. FIG. 3C shows a state in which prelasing as abnormal oscillation occurs during the oscillation of the laser light 114 before the giant pulse oscillation is started. The prelasing oscillation is a phenomenon in which the laser light leaks from part of the resonator 103 before the giant pulse oscillation. The following problem can be caused by this phenomenon. That is, the prelasing oscillation may induce local giant pulse oscillation to cause energy concentration in the resonator. In addition, the prelasing oscillation is not controlled laser light, and hence, when the prelasing oscillation occurs, the giant pulse oscillation becomes unstable. As will be described in detail below, in order to constantly keep the Q value of the resonator 103 before the giant pulse oscillation low and suppress the prelasing, control of a polarization characteristic based on a temperature change in the internal portion of the resonator is important.

### <Verification Result>

The voltage applied to the Pockels cell of the laser apparatus 110 was set to 2000 V, and the temperature of the Pockels cell was increased. The giant pulse oscillation was executed with the applied voltage of 2000 V without executing a voltage determination method of the present invention described later. As a result, the prelasing oscillation was detected using a light detector during the giant pulse oscillation. Subsequently, the measurement of the output and the intensity distribution of the laser light 114 from the laser apparatus 110 was executed. As a result, the output and the intensity distribution of the laser light 114 became unstable.

As another comparative technique that is compared with the present invention, there is a wavelength tunable laser that uses the birefringent filter as a polarization section in the resonator. The birefringent filter of this laser apparatus is a wavelength selection mechanism that utilizes the polarization characteristic. Accordingly, the prelasing oscillation caused by the change of the polarization state of the light flux in the resonator influences selectivity of the wavelength by the birefringent filter. As a result, abnormal oscillation having the wavelength different from the desired wavelength of the giant pulse oscillation occurs. For example, in the case where the giant pulse having a wavelength of 755 nm oscillates with the Pockels cell applied voltage of 2000 V, when the Pockels cell applied voltage is changed to the lower voltage side, the prelasing oscillation having the wavelength on the shorter wavelength side of about 750 nm occurs. On the other hand, when the Pockels cell applied voltage is changed to the higher voltage side, the prelasing oscillation having the wavelength on the longer wavelength side of about 760 nm occurs.

### First Embodiment

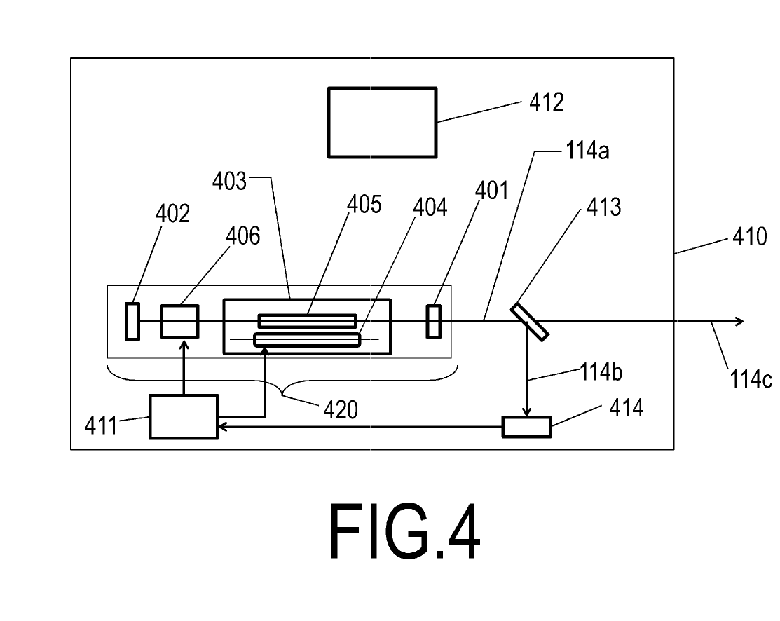

FIG. 4 is a block diagram showing a first embodiment of the laser apparatus of the present invention. A laser apparatus 410 includes a light detector 414 serving as a detector that detects light released from an output mirror 401 outside a resonator 420. Herein, the light detector 414 is an intensity sensor capable of measuring the intensity of light. A detection signal as the detection result is output to a laser controller 411, and control of a Q switch voltage is performed by the laser controller 411. The laser controller 411 can include information processing apparatuses and circuits such as a CPU, an MPU, and a memory.

A laser power supply 412 supplies power to individual blocks of the laser apparatus 410. The light detector 414 is disposed inside the laser apparatus 410, but the present invention is not limited thereto, and the light detector 414 may also be disposed outside the laser apparatus 410. In the laser apparatus 410, branch light 114b of the light flux of laser light 114a is introduced into the light detector 414, but the present invention is not limited thereto. That is, a general-purpose light sensor such as a Si sensor or the like has a sufficiently high photosensitivity. Accordingly, the configuration may appropriately detect part of the light flux of partially diffused light in an optical path of the light flux of the laser light 114a or weak transmitted light having passed through a reflection mirror 402. Note that, in the present embodiment, the laser apparatus 410 in which the alexandrite crystal is used as the light source is used, but the present invention is not limited thereto, and other light sources may also be used.

The alexandrite laser as the laser apparatus 410 of the present embodiment has a wavelength of 755 nm and a pulse repetition frequency of 20 Hz. The resonator 420 that includes the output mirror 401 and a reflection mirror 402 and a lamp house 403 that includes an alexandrite crystal 405 and a flash lamp 404 as an excitation unit (an excitation device) are disposed. The temperature of the alexandrite crystal is maintained by a circulated water apparatus having a water temperature of 75° C. A Q switch 406 that includes the Pockels cell is disposed on an optical axis between the reflection mirror 402 and the alexandrite crystal 405. A configuration is adopted in which prelasing light oscillating from the output mirror 401 is branched using a branch optical element 413 provided at a stage prior to the light detector 414 first, and branch light 114b is detected using the light detector 414 provided at a stage subsequent to the branch optical element 413.

### <Voltage Determination Method>

A method for determining an optimum applied voltage during the giant pulse oscillation that uses the laser apparatus 410 of FIG. 4 will be described in detail below. As the first step, an initial voltage of 2000 V was set as a temporary voltage applied to the Pockels cell. Next, as the second step, the presence or absence of the occurrence of the prelasing was determined by using the light detector installed outside the resonator by setting the voltage to 2000 V without allowing the giant pulse oscillation and changing the applied voltage in the range of ±500 V while continuing the application of the voltage. Specifically, the laser controller 411 changed the applied voltage stepwise from 2000 V to 1500 V. In the same manner, the applied voltage was changed stepwise from 2000 V to 2500 V. When the foregoing was executed, the prelasing occurred when the Pockels cell applied voltage was not more than 1600 V and not less than 2300 V. Next, as the third step, the mean value 1950 V (arithmetic mean) of the upper limit value as the maximum value and the lower limit value as the minimum value of the detected voltage was determined as the last applied voltage during the giant pulse oscillation. Next, as the fourth step, the giant pulse oscillation was performed with the last applied voltage of 1950 V. Note that a series of the first to fourth steps were continuously controlled using firmware.

### <Verification Result>

The measurement of the output and the light intensity distribution of the laser light of the giant pulse oscillation that used the above voltage determination method was performed. As a result, it was confirmed that, in the laser apparatus 410 of the present embodiment, the prelasing light did not oscillate immediately before the oscillation of the giant pulse in the fourth step, and the laser apparatus 410 had a laser characteristic in which the output and the light intensity distribution of the laser light of the giant pulse oscillation were stable.

### <Re-Verification of Effect of Voltage Determination Method of First Embodiment>

The effect of the voltage determination method of the present embodiment was re-verified after changing part of environmental conditions of the first embodiment. The detailed description is shown below.

### <Voltage Determination Method>

As the first step, similarly, the initial voltage of 2000 V was set as the temporary voltage applied to the Pockels cell first. Further, in order to verify the effect of the present voltage determination method, the polarization characteristic of the above verification was changed by warming the Pockels cell (in a state in which the polarization characteristic of the Q switch was affected), the following steps were performed. That is, as the second step, the presence or absence of the occurrence of the prelasing was determined by using the light detector installed outside the resonator by setting the voltage to 2000 V without allowing the giant pulse oscillation and changing the applied voltage in the range of ±500 V while continuing the application of the voltage. As a result, unlike the above verification, the prelasing occurred when the applied voltage supplied to the Pockels cell was not more than 1500 V and not less than 1900 V. Next, as the third step, the mean value 1700 V (arithmetic mean) of the upper limit value and the lower limit value of the detected voltage was determined as the last applied voltage during the giant pulse oscillation. Next, as the fourth step, the giant pulse oscillation was performed with the above last applied voltage of 1700 V. Note that the series of the first to fourth steps were continuously controlled using firmware.

### <Verification Result>

The measurement of the output and the light intensity distribution of the laser light 114a of the giant pulse oscillation that used the above voltage determination method was performed. As a result, in this experiment as well, it was confirmed that, in the laser apparatus 410 of the present embodiment, the prelasing light did not oscillate immediately before the giant pulse oscillation in the fourth step, and the laser apparatus 410 had the laser characteristic in which the output and the light intensity distribution of the laser light 114a were stable.

### <Detailed Description of Voltage Determination Method>

The method for determining the applied voltage to the Pockels cell constituting the Q switch during the giant pulse oscillation of the present invention will be described below. Firstly, as the temporary voltage, the initial voltage applied to the Pockels cell is determined. The polarization of the light flux passing through the Pockels cell is proportional to the applied voltage and the wavelength, and is determined at the time of the adjustment of the resonator.

Secondly, the initial voltage is changed while the voltage is applied to the Pockels cell, i.e., the Q switch is kept ON such that the giant pulse oscillation is not performed. Subsequently, it is determined whether or not the prelasing has occurred during the fluctuation of the applied voltage by using the light detector installed outside the resonator. The range of fluctuation of the applied voltage differs depending on the state of the resonator, and it is only necessary to cause the applied voltage to fluctuate in the range of the initial voltage ±500 V. In the case of the laser apparatus in which the polarization state of the resonator is relatively stable, the voltage fluctuation range may be ±200 V, and it is possible to make the range narrower as the resonator is more stable. Safety is confirmed by changing an external atmospheric temperature at the time of the adjustment of the resonator, and the required voltage fluctuation range is determined in advance.

Thirdly, the last applied voltage as the above finally determined applied voltage is determined. When the giant pulse oscillation is performed, the last applied voltage as the optimized applied voltage is applied to the Q switch. As described above, in the case where the prelasing does not occur even when the applied voltage is caused to fluctuate from the temporary voltage, the temporary voltage may be used as the last applied voltage. In the case where the prelasing has occurred, the voltage at which the prelasing has not occurred is set as the last applied voltage. For example, it is assumed that the initial applied voltage is V_initial and the last applied voltage is V_last. In the case where the prelasing has occurred at the time of the voltage change of +200 V and at the time of voltage change of −500 V, the voltage range in which the prelasing does not occur is V_initial −500 [V]<V_last<V_initial +200 [V]. Herein, as the last applied voltage V_last, it is preferable to use the last applied voltage V_last=V_initial −150 [V] as the arithmetic mean of the lower limit value (V_initial −500 [V]) and the upper limit value (V_initial +200 [V]). In the case where the prelasing does not occur at the limit value of the voltage fluctuation range, the limit value is set to the upper limit value or the lower limit value. It is possible to perform the stable laser oscillation by successively executing the series of the first to third steps for causing the giant pulse oscillation. The present applied voltage determination method is performed in a short period of time of not more than several tens of seconds, more preferably several hundreds of ms such that the laser resonance state does not change.

### <Effect>

The present invention is suitably used especially in the flash lamp-excitation alexandrite laser. The gain coefficient of the alexandrite crystal increases in proportion to the temperature, and the laser output is thereby increased. Accordingly, by increasing the temperature of the laser crystal to a high temperature by using the circulated water of 60° C. to 80° C., the laser output is increased. On the other hand, the temperature of the internal portion of the resonator is likely to be unstable. By using the present invention, it is possible to obtain the stable giant pulse oscillation in which the occurrence of the prelasing is reduced even without using a complicated temperature stabilization mechanism in the resonator.

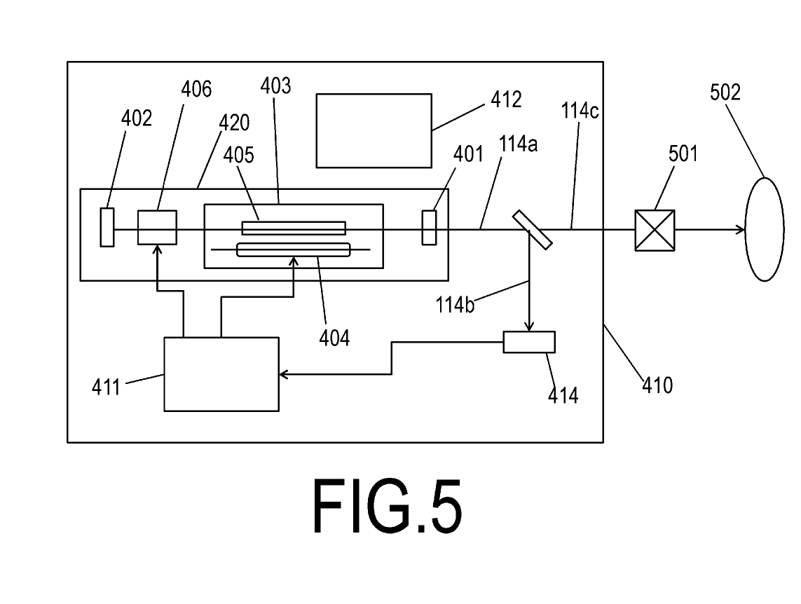

With the foregoing, when the object information acquiring apparatus shown in FIG. 5 is produced by using the laser apparatus of the present invention, it is possible to stably execute the photoacoustic measurement that uses the laser light having the large output. Therefore, it is possible to acquire an excellent object image having high contrast.

### <Object Measurement>

FIG. 5 is a block diagram including the laser apparatus of the present invention and the object. Herein, a configuration is adopted in which a shutter 501 is provided between the laser apparatus 410 of FIG. 4 and an object 502, and the object 502 is irradiated with laser light 114c via the shutter 501. Elements common to the laser apparatus of FIG. 4 are designated by the same reference numerals and the description thereof will be omitted. In the present configuration, when the prelasing light is detected, the shutter 501 is closed and the irradiation of the object 502 with the laser light 114c is prevented. The branch light 114b is guided to the detector 414, and the prelasing light is detected using the detector 414. Note that the shutter 501 may also be disposed inside the laser apparatus 410.

### Second Embodiment

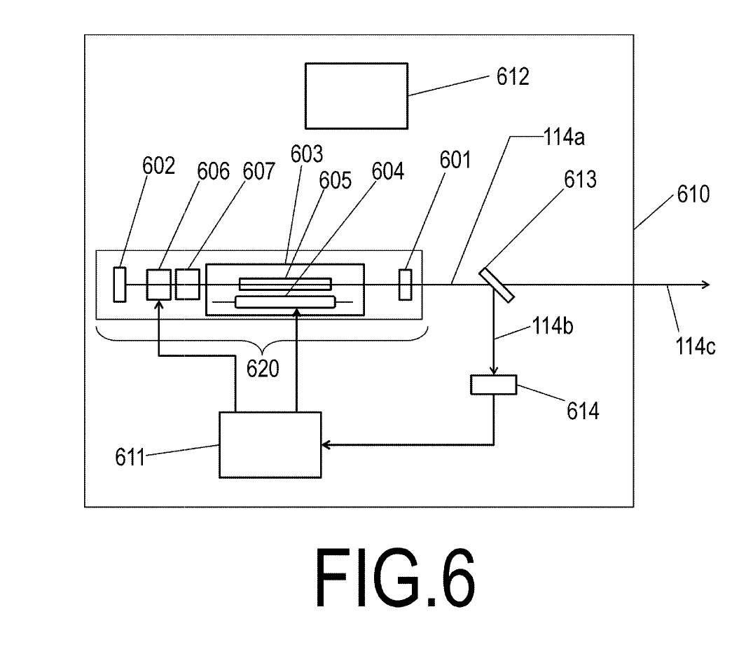

FIG. 6 is a block diagram showing a second embodiment of the laser apparatus of the present invention. In the present embodiment, the alexandrite laser capable of oscillation with the wavelength of 785 nm is used as the light source. A resonator 620 that includes an output mirror 601 and a reflection mirror 602 and a lamp house 603 that includes an alexandrite crystal 605 and a flash lamp 604 as the excitation unit are disposed. The temperature of the alexandrite crystal is maintained by the circulated water apparatus having a water temperature of 75° C. By disposing a Q switch 606 that includes the Pockels cell and a birefringent filter 607 as the wavelength selection element on the optical axis between the reflection mirror 602 and the alexandrite crystal 605, the wavelength of the output laser light 114c can be variably controlled. The wavelength of the prelasing light is detected by branching the prelasing light oscillating from the output mirror 601 using a branch optical element 613, replacing the light detector 414 of the first embodiment with a wave meter 614, and guiding the branch light 114b to the wave meter 614. A laser controller 611 controls the Q switch voltage, and a laser power supply 612 supplies power to individual blocks of a laser apparatus 610. Note that the depiction of wiring of the laser power supply 612 or the like is omitted. Note that the laser controller 611 can include information processing apparatuses and circuits such as a CPU, an MPU, and a memory.

### <Voltage Determination Method>

The method for determining the last applied voltage during the giant pulse oscillation that uses the laser apparatus 610 of the present embodiment will be described in detail below. As the first step, the initial voltage of 2300 V was set as the temporary voltage applied to the Pockels cell first. Next, the polarization characteristic was changed by warming the Pockels cell (the gain coefficient of the alexandrite crystal increases in proportion to the temperature, and the laser output is thereby increased. Accordingly, by increasing the temperature of the laser crystal to a high temperature by using the circulated water of 60° C. to 80° C., the laser output is increased. However, on the other hand, the temperature of the internal portion of the resonator is likely to be unstable. As a result, the characteristic of the Q switch is changed, and the prelasing becomes likely to occur. This state is simulated.), and the following steps were performed. That is, as the second step, the presence or absence of the occurrence of the prelasing was determined by using the wave meter 614 installed outside the resonator 620 by setting the voltage to 2300 V without allowing the giant pulse oscillation and changing the applied voltage in the range of ±500 V while continuing the application of the voltage. As a result, the oscillation having a wavelength of 778 nm corresponding to the prelasing occurred when the applied voltage to the Pockels cell was not more than 1900 V (the lower limit value). In addition, the oscillation having a wavelength of 792 nm corresponding to the prelasing occurred when the applied voltage to the Pockels cell was not less than 2600 V (the upper limit value). Next, as the third step, the mean value 2250 V (arithmetic means) of the upper limit value and the lower limit value of the detected voltage was determined as the last applied voltage during the giant pulse oscillation. Next, as the fourth step, the giant pulse oscillation was performed with the above last applied voltage. Note that, similarly to the above embodiment, the series of the first to fourth steps were continuously controlled using firmware.

### <Verification Result>

The oscillation wavelength of the giant pulse oscillation that used the above voltage determination method was the desired wavelength of 785 nm, and it was confirmed that the laser apparatus 610 had the stable laser characteristic as the result of measurement of the output and the light intensity distribution of the laser light 114c.

### <Effect>

With the foregoing, according to the laser apparatus 610 of the present invention, it is possible to stably output the giant pulse having the desired wavelength.

### Third Embodiment

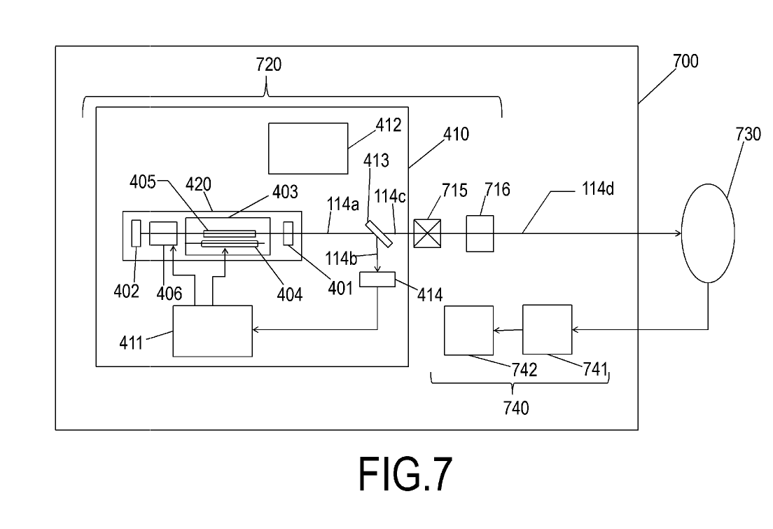

FIG. 7 is a view showing a third embodiment of the laser apparatus of the present invention. The embodiment in the case where the laser apparatus 410 of the first embodiment is applied to an object information acquiring apparatus 700 will be described by using FIG. 7. Note that the laser apparatus 410 of the first embodiment is the alexandrite laser, and its output is the laser light 114c having a wavelength of 755 nm and a pulse repetition frequency of 20 Hz. The object information acquiring apparatus 700 roughly includes an input system 720 and an output system 740.

The input system 720 includes the laser apparatus 410, a shutter 715, and an illumination optical section 716 as an irradiation section (an irradiation device). The light flux of the laser light 114a caused to oscillate from the resonator 420 as the light source passes through a light transmission optical system such as a fiber or an articulated arm. Thereafter, the laser light 114c emitted from the laser apparatus 410 is formed into a desired shape by using the illumination optical section 716 such as a lens and a diffuser. An object 730 is irradiated with formed irradiation light 114d. In the light transmission optical system, a timing trigger is appropriately disposed such that the irradiation light 114d can be applied at a timing required for signal processing in a signal processing section (a signal processor) 742 as an acquisition section (an acquisition device) described later.

The output system 740 includes an acoustic wave receiver 741 and the signal processing section 742. The photoacoustic wave that is generated inside the object 730 and propagates is received by an ultrasonic probe in the acoustic wave receiver 741. The ultrasonic probe sends an analog electric signal as the reception result to the signal processing section 742 via a signal line. The sent analog electric signal is converted to a digital photoacoustic signal in the signal processing section 742, and image formation (image reconstruction) is performed based on the photoacoustic signal. Subsequently, data generated by the image reconstruction is output as image information and biological information. Any probe can be used as the above probe as long as the probe can receive the acoustic wave such as a probe that uses piezoelectricity, a probe that uses light resonance, or a probe that uses a change of a capacitance. The probe of the present embodiment is preferably a probe in which a plurality of reception elements are disposed one-dimensionally or two-dimensionally, or a probe in which the plurality of reception elements are spirally disposed at a bottom of a bowl-shaped fixed component. By using the multidimensional array element, it is possible to receive the acoustic wave at a plurality of positions simultaneously. The signal processing section 742 is configured by an information processing apparatus such as a computer and a circuit, and performs processing of the electric signal and arithmetic calculation. The signal processing section 742 has an A/D converter or the like as a conversion section that converts the electric signal obtained by the probe included in the acoustic wave receiver 741 from the analog signal to the digital signal on the side of the probe in the signal processing section 742. In addition, the signal processing section 742 performs parallel processing that processes a plurality of signals simultaneously. With this, it is possible to increase a signal processing speed. The digital signal converted by the A/D converter or the like is stored in a memory in the signal processing section 742 and, by performing processing such as back projection of a time domain or the like based on the stored digital signal, object information such as an optical characteristic value distribution or the like is generated.

Herein, the periphery of the resonator 420 will be described more specifically. The resonator 420 that includes the output mirror 401 and the reflection mirror 402 and the lamp house 403 that includes the flash lamp 404 as the unit for exciting the alexandrite crystal 405 are disposed. The temperature of the alexandrite crystal is maintained by the circulated water apparatus having a water temperature of 75° C. The Q switch 406 that includes the Pockels cell is disposed on the optical axis between the reflection mirror 402 and the alexandrite crystal 405. The prelasing light oscillating from the output mirror 401 is branched using the branch optical element 413 and detected using the light detector 414. The input system is configured by the alexandrite laser, i.e., the laser apparatus 410, the shutter 715, and the illumination optical section 716 as the irradiation section that uses the diffuser. The output system 740 is configured by the acoustic wave receiver 741 and the signal processing section 742.

### <Voltage Determination Method>

The method for determining the applied voltage during the giant pulse oscillation that uses the laser apparatus 410 of the first embodiment of FIG. 7 will be described in detail below. As the first step, the initial voltage of 2000 V was set as the temporary voltage applied to the Pockels cell first. As the second step, the presence or absence of the occurrence of the prelasing was determined by using the light detector 414 installed outside the resonator 420 by setting the voltage to 2000 V without allowing the giant pulse oscillation and changing the applied voltage in the range of ±500 V while continuing the application of the voltage. As a result, the prelasing occurred when the applied voltage to the Pockels cell was not more than 1600 V and not less than 2300 V. Next, as the third step, the mean value 1950 V (arithmetic means) of the upper limit value (2300 V) and the lower limit value (1600 V) of the detected voltage was determined as the last applied voltage during the giant pulse oscillation. Next, as the fourth step, the giant pulse oscillation was performed with the above last applied voltage. The series of the first to fourth steps were continuously controlled.

### <Result of Object Information Acquisition>

The object phantom 730 of which the shape was fixed using an object holding section (not shown) was irradiated with the laser light 114d caused to oscillate by using the above voltage determination method. The photoacoustic wave that was generated in the object 730 based on the irradiation and propagated was detected using a cMUT probe using the change of the capacitance in the acoustic wave receiver 741. As the probe, the probe spirally disposed at the bottom of a bowl-shaped fixed member was used. The electric signal obtained by the probe was converted from the analog signal to the digital signal in the signal processing section 742 and the photoacoustic signal was thereby generated, and the image reconstruction was performed using the time domain based on the signal. Based on the reconstruction image, the object information such as the image information and the optical characteristic value distribution was output.

### <Effect>

The occurrence of the prelasing is reduced by using the object information acquiring apparatus of the present embodiment, and it is possible to stably perform the irradiation with the laser light having the desired wavelength and intensity. As a result, accurate object information was acquired.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment (s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2014-137597, filed on Jul. 3, 2014, which is hereby incorporated by reference herein in its entirety.

What is claimed is:

1. An object information acquiring apparatus comprising:
a laser resonator configured to include two reflection bodies and a Q switch provided between the two reflection bodies and determining a Q value based on a voltage to be applied;
an excitation section configured to optically excite a laser medium;
a detector configured to detect light output from one of the reflection bodies;
a controller configured to control the laser resonator and determine a value of the voltage based on a detection result of the detector and applies the voltage to the Q switch;
a receiver configured to receive an acoustic wave propagating from an object based on irradiation of the object with laser light output from one of the reflection bodies; and
an acquisition section configured to acquire information relate to the object based on a reception result of the receiver.

2. The object information acquiring apparatus according to claim 1, further comprising a branch optical element configured to be provided at a stage prior to the detector and guides part of the laser light output from one of the reflection bodies to the detector, wherein
the detector performs the detection based on part of the laser light.

3. The object information acquiring apparatus according to claim 1, wherein
the light output from one of the reflection bodies includes prelasing light.

4. The object information acquiring apparatus according to claim 3, wherein
the controller acquires a minimum value and a maximum value of the voltage to be applied when the prelasing light is not generated based on the detection result, and determines the value of the voltage on the basis of the acquired result.

5. The object information acquiring apparatus according to claim 4, wherein
the determined value of the voltage is a value between the minimum value and the maximum value.

6. The object information acquiring apparatus according to claim 5, wherein
the determined value of the voltage is a mean value of the minimum value and the maximum value.

7. The object information acquiring apparatus according to claim 1, wherein
the detector detects an intensity of light.

8. The object information acquiring apparatus according to claim 1, further comprising a polarization section configured to polarize the laser light, wherein
the detector detects a wavelength of light.

9. The object information acquiring apparatus according to claim 8, wherein
the polarization section is provided between the Q switch and the laser medium.

10. The object information acquiring apparatus according to claim 9, wherein
a wavelength of the laser light is variably controlled.

11. A laser apparatus comprising:
a laser resonator configured to include two reflection bodies and a Q switch provided between the two reflection bodies and determining a Q value based on a voltage to be applied;
an excitation section configured to optically excite a laser medium;
a detector configured to detect light output from one of the reflection bodies; and
a controller configured to control the laser resonator and determine a value of the voltage based on a detection result of the detector and applies the voltage to the Q switch.
